(19) United States
(12) Patent Application Publication
(10) Pub. No.: US 2017/0253918 A1
(43) Pub. Date: Sep. 7, 2017

(54) COMBINING PROTEIN BARCODING WITH EXPANSION MICROSCOPY FOR IN-SITU, SPATIALLY-RESOLVED PROTEOMICS

(71) Applicant: Expansion Technologies, Boston, MA (US)

(72) Inventor: Richie E. KOHMAN, Cambridge, MA (US)

(21) Appl. No.: 15/446,005

(22) Filed: Mar. 1, 2017

Related U.S. Application Data
(60) Provisional application No. 62/301,871, filed on Mar. 1, 2016.

(51) Int. Cl. C12Q 1/68; G01N 21/64; G01N 1/34; G01N 1/30; G01N 1/28

(52) U.S. Cl. CPC C12Q 1/6816; C12Q 1/6806; C12Q 1/6804; G01N 1/30; G01N 1/286; G01N 1/34; G01N 21/6428; G01N 2021/6439

(57) ABSTRACT

This invention relates to imaging, such as by expansion microscopy, labelling, and analyzing biological samples, such as cells and tissues, as well as reagents and kits for doing so.

### CROSS REFERENCE TO RELATED APPLICATIONS

This application claims priority to U.S. Provisional Application 62/301,871, filed Mar. 1, 2016, which is hereby incorporated therein in its entirety.

### FIELD OF THE INVENTION

The invention relates to imaging, such as by expansion microscopy, labelling, and analyzing biological samples, such as cells and tissues, as well as reagents and kits for doing so.

### BACKGROUND OF THE INVENTION

In expansion microscopy (ExM), 3-dimensional imaging with nanoscale precision is performed on cells and tissues. This is accomplished by physically expanding the biological sample using a dense polymer matrix (FIG. 1). The first step of this process involves treating the tissue with a fluorescent protein-binding-group (typically an antibody) that selectively binds to the protein being analyzed. Next the sample is infused with a monomer solution that permeates into the tissue. Free radical polymerization of this solution creates a polymer network that is physically connected to the protein-binding-groups through customized bioconjugation chemistry. Lastly, the tissue is digested and the hydrogel (and fluorescent dyes) expands uniformly. The result is a polymer network that contains fluorescent dyes where the target proteins were located. This process has many advantages. Notably, it allows pseudo super-resolution imaging with conventional confocal microscopy because the imaging targets are no longer diffraction limited. Additionally, the tissue digestion clears the sample allowing imaging deep into thick tissues samples.

Critical to the success of the ExM process is the ability to physically connect the fluorescent protein-binding-groups to the polymer network. Current ExM attachment chemistry uses a trifunctional, double-stranded DNA linker to accomplish this. Because the tissue digestion enzymes are also capable of digesting the antibodies typically used as protein-binding-groups, the fluorescent dyes must be attached to the DNA and not the antibody. Also needed is the presence of a chemical group that can polymerize into the gel matrix (shown in FIG. 2 as a methacrylamide group on the DNA). Current examples of ExM use the chemical arrangement shown in FIG. 2 where one strand of DNA is connected to the protein-binding-group while the complementary strand possesses both the dye and the polymerizable group. Using this strategy, cells and brain tissue were successfully stained with up to 3 different protein-binding-groups, expanded, and imaged (Chen et al., Science 347:543 (2015)). However, because the number of fluorescent dyes that can be used is small (typically <6), this strategy is limited to imaging only a small number of proteins per sample. Additionally, the polymerization process dampens the fluorescence of the dyes, which are permanently connected to the gel matrix. These problems can be overcome by utilizing an improved bioconjugation strategy. By rearranging the location of the three chemical groups (dye, gel binding group, and protein-binding-group) on the DNA linker, previous limitations in protein imaging can be overcome.

### SUMMARY OF THE INVENTION

In one aspect, provided herein are methods of labeling a biological sample, the methods comprising the steps of: contacting the sample with at least one binding composition under conditions to selectively recognize a target biomolecule, wherein the binding composition comprises a double-stranded nucleic acid having a first strand comprising a first sequence and a second strand comprising a second sequence that is complementary to the first sequence, wherein the first strand is operably linked to an affinity tag for the target biomolecule; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; and dialyzing the sample in water to expand the polyelectrolyte gel. In some embodiments, the first strand is also operably linked to a first detectable label, and the second strand is operably linked to a polyelectrolyte gel binding moiety. In some embodiments, the first strand is also operably linked to a polyelectrolyte gel binding moiety, and the second stand is operably linked to a first detectable label. In some embodiments, the methods further comprise the step of: removing the nucleic acid strand unconjugated to the polyelectrolyte gel after covalently conjugating the polyelectrolyte gel binding moiety to it; and hybridizing a nucleic acid probe operably linked to a detectable label and comprising a probe sequence that is complementary to the sequence of the nucleic acid strand conjugated to the polyelectrolyte gel.

In another aspect, provided herein are methods of labeling a biological sample, the methods comprising the steps of: contacting the sample with at least one binding composition under conditions to selectively recognize a target biomolecule, wherein the binding composition comprises a single-stranded nucleic acid comprising a first sequence, wherein the single-stranded nucleic acid is operably linked to (i) an affinity tag for the target biomolecule, and (ii) a polyelectrolyte gel binding moiety, and wherein the affinity tag is operably linked to a first detectable label; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; and dialyzing the sample in water to expand the polyelectrolyte gel. In some embodiments, the methods further comprise the step of: hybridizing a nucleic acid probe operably linked to a detectable label and comprising a probe sequence that is complementary to the sequence of the nucleic acid strand conjugated to the polyelectrolyte gel.

In another aspect, provided herein are methods of labeling a biological sample, the methods comprising the steps of: contacting the sample with at least one binding composition under conditions to selectively recognize a target biomolecule, wherein the binding composition comprises a nucleic acid operably linked to (i) an affinity tag for the target biomolecule and (ii) operably linked to a polyelectrolyte gel binding moiety, and comprising a first sequence; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; dialyzing the sample in water to expand the polyelectrolyte gel; and hybridizing a nucleic acid probe operably linked to a detectable label and comprising a probe sequence that is complementary to the first sequence. In some embodiments, the nucleic acid is a double-stranded nucleic acid having a first strand operably linked to the affinity tag and comprising the first sequence, and a second strand operably linked to the polyelectrolyte gel binding moiety and comprising a second sequence that is complementary to the first sequence. In some embodiments, the binding composition comprises a single-stranded nucleic acid is operably linked to both the affinity tag and the polyelectrolyte gel binding moiety.

In another aspect, provided herein are methods of imaging a biological sample, the methods comprising the steps of: labeling the sample with a detectable label as described herein, and obtaining an image of the sample by detecting the detectable label after expanding the polyelectrolyte gel. In some embodiments, an image of the sample is also obtained before expanding the polyelectrolyte gel. In some embodiments, images are obtained by confocal microscopy.

In another aspect, provided herein are methods of analyzing a biological sample, the methodw comprising the steps of: (a) contacting the sample with a set of adapters that selectively recognize a set of target biomolecules under conditions where the adapters selectively recognize the target biomolecules, wherein each adapter comprises a nucleic acid operably linked to an affinity tag specific for one of the target biomolecules and operably linked to a polyelectrolyte gel binding moiety and having a sequence specific for that target biomolecule; (b) contacting the sample with a solution comprising monomers of a polyelectrolyte gel; (c) by free radical polymerization, polymerizing the monomers to form the polyelectrolyte gel and covalently conjugate the polyelectrolyte gel binding moiety to the polyelectrolyte gel; (d) proteolytically digesting the sample; (e) dialyzing the sample in water to expand the polyelectrolyte gel; and (f) for each target biomolecule, hybridizing a nucleic acid probe operably linked to a fluorescent label and comprising a probe sequence complementary to the sequence specific for that target biomolecule, and detecting the fluorescent label. In some embodiments, the nucleic acid is a double stranded nucleic acid comprising a first strand operably linked to the affinity tag and having a sequence specific for that target biomolecule, and a second strand comprising a sequence complementary to the sequence specific for that target biomolecule and operably linked to the polyelectrolyte gel binding moiety. In some embodiments, the nucleic acid is a single-stranded nucleic acid having a sequence specific for that target biomolecule and operably linked to both the affinity tag and the polyelectrolyte gel binding moiety. In some embodiments, the fluorescent label for multiple targets uses the same fluorophore for that set of targets.

In another aspect, provided herein are reagents (e.g., the binding compositions, adapters, nucleic acid probes) and kits for use in the methods described herein.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1. Schematic depiction of tissue processing performed in expansion microscopy (ExM).

FIG. 2. General attachment strategy used for expansion microscopy.

FIG. 3. With an improved bioconjugation strategy, a large number of proteins can be barcoded and imaged within a single tissue sample.

FIG. 4. Alternate variations of the bioconjugation chemistry, according to certain embodiments described herein.

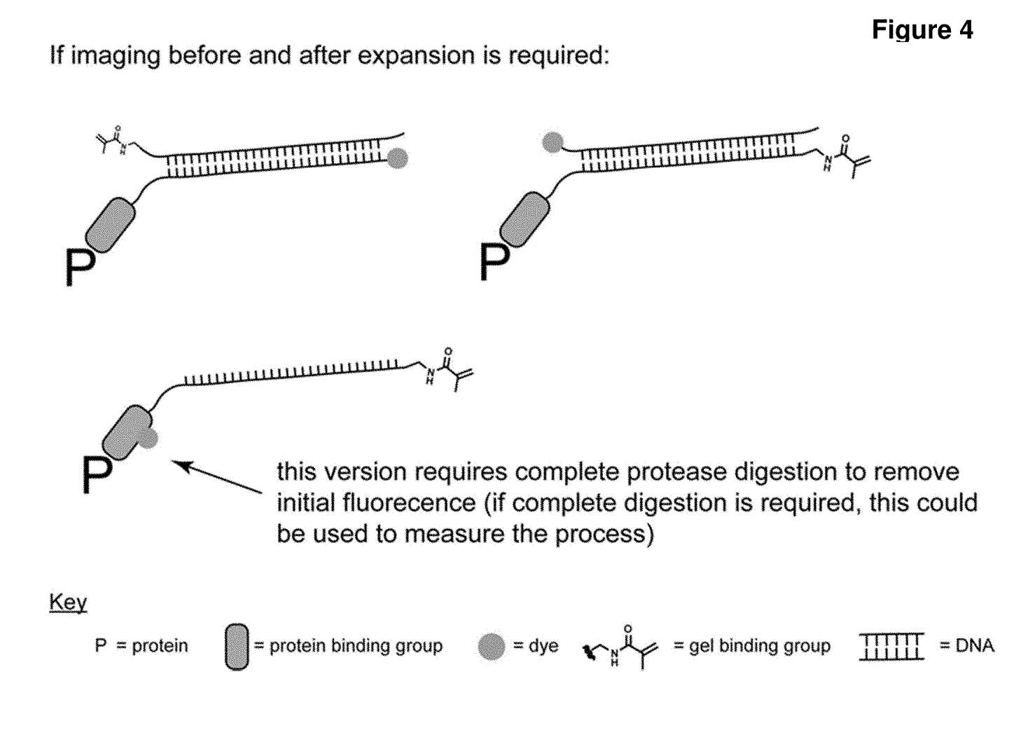

FIG. 5. Attachment chemistry, according to certain embodiments described herein, when only expanded samples need to be imaged.

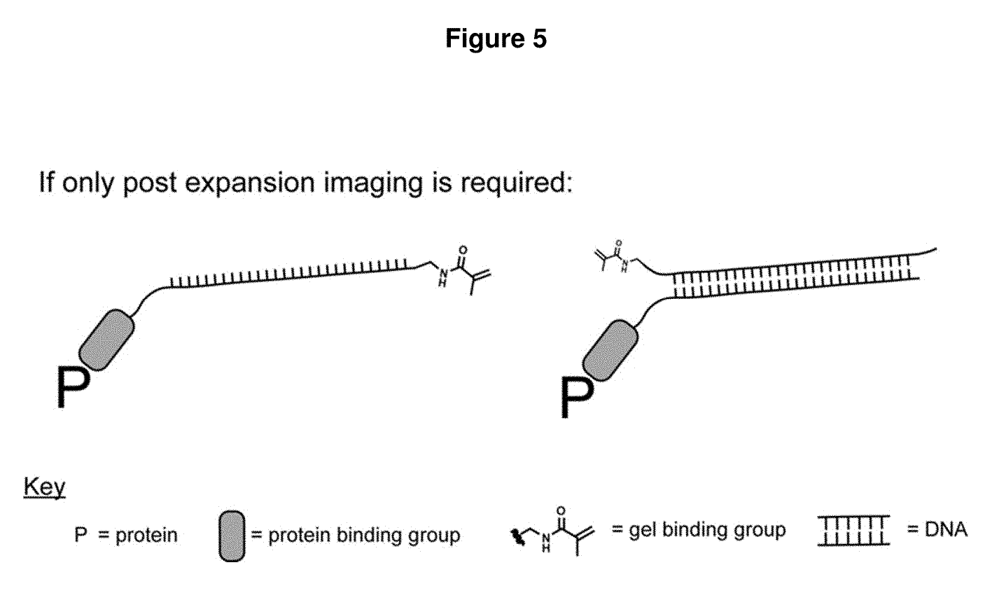

FIG. 6. Mouse brain slices stained for the parvalbumin protein with antibodies containing an oligonucleotide barcode as well as a polymerizable handle and hybridized to an oligonucleotide complementary to the barcode containing a green dye (left column) or a red dye (middle column), as well as an overlay of the images (right column) The bright line in the green image is a light artifact due to a crack in the gel.

### DETAILED DESCRIPTION OF THE INVENTION

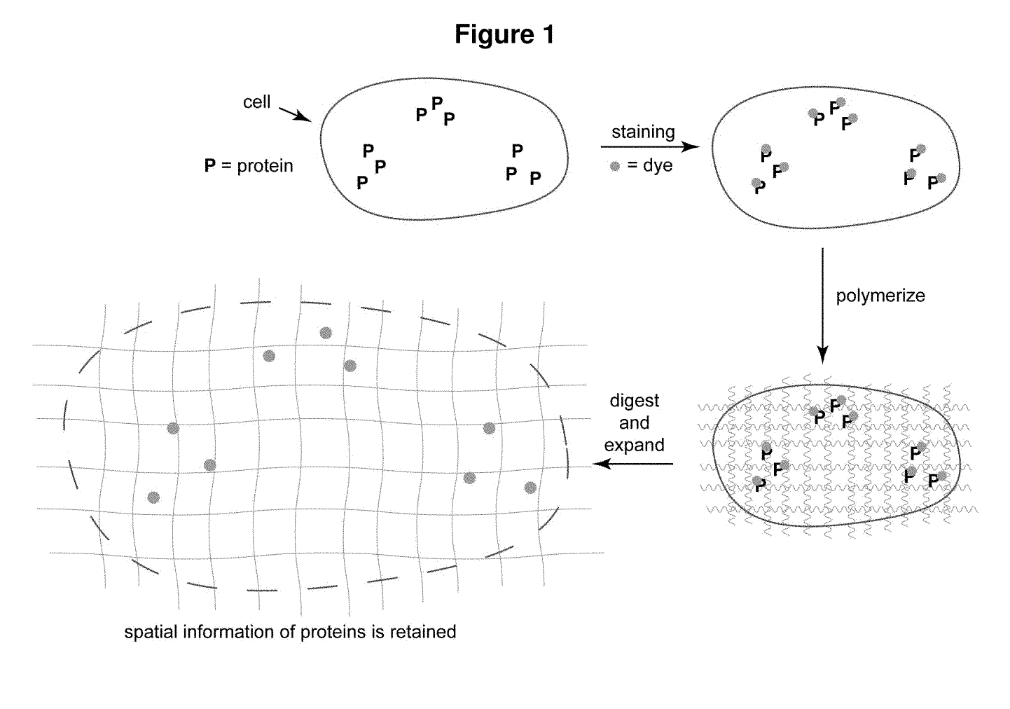

In expansion microscopy (ExM), 3-dimensional imaging with nanoscale precision is performed on cells and tissues. This is accomplished by physically expanding the biological sample using a dense polymer matrix (FIG. 1). The first step of this process involves treating the tissue with a fluorescent protein-binding-group (typically an antibody) that selectively binds to the protein being analyzed. Next the sample is infused with a monomer solution that permeates into the tissue. Free radical polymerization of this solution creates a polymer network that is physically connected to the protein-binding-groups through customized bioconjugation chemistry. Lastly, the tissue is digested and the hydrogel (and fluorescent dyes) expands uniformly. The result is a polymer network that contains fluorescent dyes where the target proteins were located. This process has many advantages. Notably, it allows pseudo super resolution imaging with conventional confocal microscopy because the imaging targets are no longer diffraction limited. Additionally, the tissue digestion clears the sample allowing imaging deep into thick tissues samples.

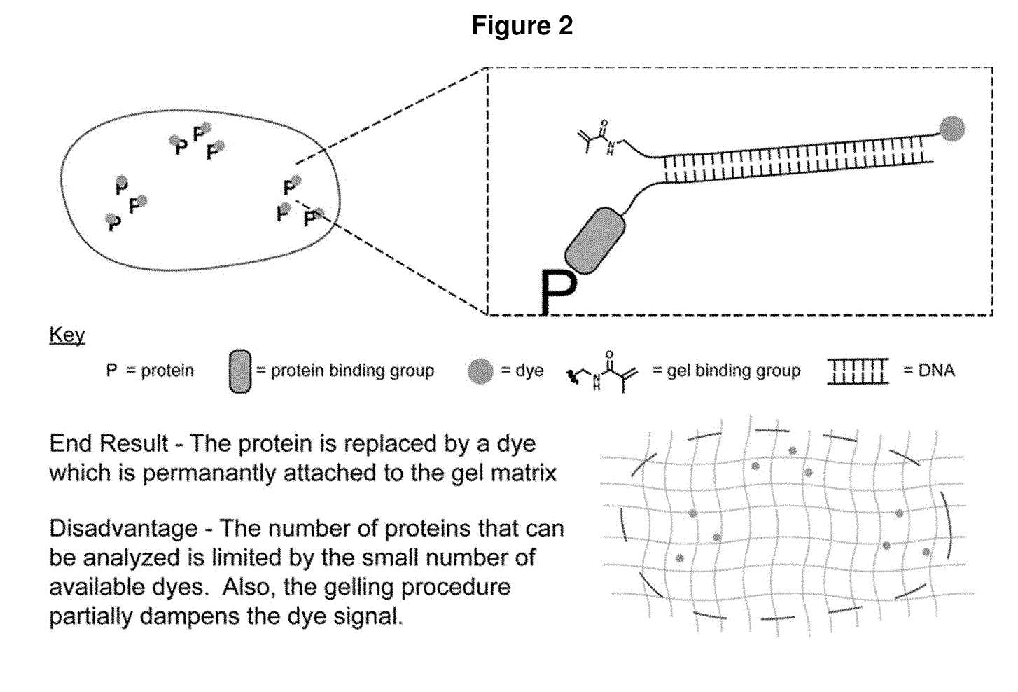

Critical to the success of the ExM process is the ability to physically connect the fluorescent protein-binding-groups to the polymer network. Current ExM attachment chemistry uses a trifunctional, double-stranded DNA linker to accomplish this. Because the tissue digestion enzymes are also capable of digesting the antibodies typically used as protein-binding-groups, the fluorescent dyes must be attached to the DNA and not the antibody. Also needed is the presence of a chemical group that can polymerize into the gel matrix (shown here as a methacrylamide group) on the DNA. Current examples of ExM use the chemical arrangement shown in FIG. 2 where one strand of DNA is connected to the protein-binding-group while the complementary strand possesses both the dye and the polymerizable group. Using this strategy, cells and brain tissue were successfully stained with up to 3 different protein-binding-groups, expanded, and imaged. However, because the number of fluorescent dyes that can be used is small (typically <6), this strategy is limited to imaging only a small number of proteins per sample. Additionally, the polymerization process dampens the fluorescence of the dyes which are permanently connected to the gel matrix. These problems can be overcome by utilizing an improved bioconjugation strategy.

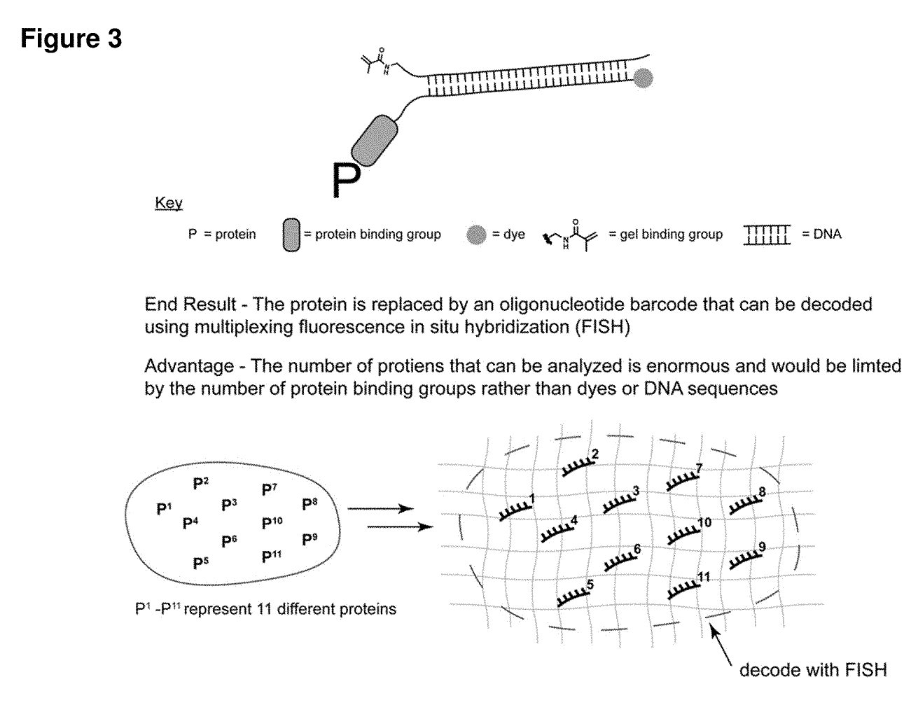

By rearranging the location of the three necessary chemical groups (dye, gel binding group, and protein-binding-group) on the DNA linker, the previous limitations in protein imaging can be overcome. FIG. 3 shows benefits of this improved strategy. In this example the dye is no longer attached to the same DNA strand as the gel binding group. The consequence is that the final polymer matrix is physically connected to a stand of DNA with a defined sequence (and no dye). Whereas the previous approach replaced the target protein with a dye that could be imaged, this improved strategy replaces the target protein with a DNA barcode. This barcode can be decoded in a subsequent step using multiplexed fluorescence in situ hybridization (FISH) which is not limited by the number of available fluorescent dyes. This modification in chemistry can allow the simultaneous tagging of many proteins in the same sample because each protein can be given a unique barcode. The small number of dyes is no longer limiting and the maximum number of proteins that can be imaged is limited now by the number of available protein-binding-groups. Additionally, because the DNA strand attached to the dye is not bound to the polymer matrix, the loss in fluorescence observed during polymerization is irrelevant because the dye-containing strand can be removed. Imaging of the barcode can be done later with FISH.

In one aspect, provided herein are methods of labeling a biological sample, the methods comprising the steps of: contacting the sample with at least one binding composition under conditions to selectively recognize a target biomolecule, wherein the binding composition comprises a double-stranded nucleic acid having a first strand comprising a first sequence and a second strand comprising a second sequence that is complementary to the first sequence, wherein the first strand is operably linked to an affinity tag for the target biomolecule; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; and dialyzing the sample in water to expand the polyelectrolyte gel. In some embodiments, the first strand is also operably linked to a first detectable label, and the second strand is operably linked to a polyelectrolyte gel binding moiety. In some embodiments, the first strand is also operably linked to a polyelectrolyte gel binding moiety, and the second stand is operably linked to a first detectable label. In some embodiments, the methods further comprise the step of: removing the nucleic acid strand unconjugated to the polyelectrolyte gel after covalently conjugating the polyelectrolyte gel binding moiety to it; and hybridizing a nucleic acid probe operably linked to a detectable label and comprising a probe sequence that is complementary to the sequence of the nucleic acid strand conjugated to the polyelectrolyte gel.

In another aspect, provided herein are methods of labeling a biological sample, the methods comprising the steps of: contacting the sample with at least one binding composition under conditions to selectively recognize a target biomolecule, wherein the binding composition comprises a single-stranded nucleic acid comprising a first sequence, wherein the single-stranded nucleic acid is operably linked to (i) an affinity tag for the target biomolecule, and (ii) a polyelectrolyte gel binding moiety, and wherein the affinity tag is operably linked to a first detectable label; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; and dialyzing the sample in water to expand the polyelectrolyte gel. In some embodiments, the methods further comprise the step of: hybridizing a nucleic acid probe operably linked to a detectable label and comprising a probe sequence that is complementary to the sequence of the nucleic acid strand conjugated to the polyelectrolyte gel.

In another aspect, provided herein are methods of labeling a biological sample, the methods comprising the steps of: contacting the sample with at least one binding composition under conditions to selectively recognize a target biomolecule, wherein the binding composition comprises a nucleic acid operably linked to (i) an affinity tag for the target biomolecule and (ii) operably linked to a polyelectrolyte gel binding moiety, and comprising a first sequence; contacting the sample with a solution comprising monomers of a polyelectrolyte gel; by free radical polymerization, polymerizing the monomers to form the polyelectrolyte gel and covalently conjugate the polyelectrolyte gel binding moiety to the polyelectrolyte gel; proteolytically digesting the sample; dialyzing the sample in water to expand the polyelectrolyte gel; and hybridizing a nucleic acid probe operably linked to a detectable label and comprising a probe sequence that is complementary to the first sequence. In some embodiments, the nucleic acid is a double-stranded nucleic acid having a first strand operably linked to the affinity tag and comprising the first sequence, and a second strand operably linked to the polyelectrolyte gel binding moiety and comprising a second sequence that is complementary to the first sequence. In some embodiments, the binding composition comprises a single-stranded nucleic acid is operably linked to both the affinity tag and the polyelectrolyte gel binding moiety.

In another aspect, provided herein are methods of imaging a biological sample, the methods comprising the steps of: labeling the sample with a detectable label as described herein, and obtaining an image of the sample by detecting the detectable label after expanding the polyelectrolyte gel. In some embodiments, an image of the sample is also obtained before expanding the polyelectrolyte gel. In some embodiments, images are obtained by confocal microscopy.

In another aspect, provided herein are methods of analyzing a biological sample, the methodw comprising the steps of: (a) contacting the sample with a set of adapters that selectively recognize a set of target biomolecules under conditions where the adapters selectively recognize the target biomolecules, wherein each adapter comprises a nucleic acid operably linked to an affinity tag specific for one of the target biomolecules and operably linked to a polyelectrolyte gel binding moiety and having a sequence specific for that target biomolecule; (b) contacting the sample with a solution comprising monomers of a polyelectrolyte gel; (c) by free radical polymerization, polymerizing the monomers to form the polyelectrolyte gel and covalently conjugate the polyelectrolyte gel binding moiety to the polyelectrolyte gel; (d) proteolytically digesting the sample; (e) dialyzing the sample in water to expand the polyelectrolyte gel; and (f) for each target biomolecule, hybridizing a nucleic acid probe operably linked to a fluorescent label and comprising a probe sequence complementary to the sequence specific for that target biomolecule, and detecting the fluorescent label. In some embodiments, the nucleic acid is a double stranded nucleic acid comprising a first strand operably linked to the affinity tag and having a sequence specific for that target biomolecule, and a second strand comprising a sequence complementary to the sequence specific for that target biomolecule and operably linked to the polyelectrolyte gel binding moiety. In some embodiments, the nucleic acid is a single-stranded nucleic acid having a sequence specific for that target biomolecule and operably linked to both the affinity tag and the polyelectrolyte gel binding moiety. In some embodiments, the fluorescent label for multiple targets uses the same fluorophore for that set of targets.

In another aspect, provided herein are reagents (e.g., the binding compositions, adapters, nucleic acid probes) and kits for use in the methods described herein.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In each pair, the light and heavy chain variable regions (V_{L} and V_{H}) are together responsible for binding to an antigen, and the constant regions (C_{L}, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains V_{H}, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to V_{H}, Cγ1, Cγ2, Cγ3, V_{L}, and C_{L}.

Depending on the amino acid sequence of the constant domain of their heavy chains, intact antibodies can be assigned to different “classes.” There are five-major classes (isotypes) of intact antibodies: IgA, IgD, IgE, IgG, and IgM, and several of these may be further divided into “subclasses,” e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

The terms “antibody” or “antigen-binding fragment” respectively refer to intact molecules as well as functional fragments thereof, such as Fab, a scFv-Fc bivalent molecule, F(ab′)₂, and Fv that are capable of specifically interacting with a desired target. In some embodiments, the antigen-binding fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the variable region of the light chain and the variable region of the heavy chain expressed as two chains;
(5) Single chain antibody (“SCA”), a genetically engineered molecule containing the variable region of the light chain and the variable region of the heavy chain, linked by a suitable polypeptide linker as a genetically fused single chain molecule; and
(6) scFv-Fc, is produced by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tandem scFv, a scFv-Fc bivalent molecule, an Fab, Fab′, Fv, F(ab′)₂ or an antigen binding scaffold (e.g., affibody, monobody, anticalin, DARPin, Knottin, etc.).

As used herein, the terms “binds” or “binding” or grammatical equivalents, refer to compositions, directly or indirectly, having affinity for each other. “Specific binding” is where the binding is selective between two molecules. A particular example of specific binding is that which occurs between an antibody and an antigen. Typically, specific binding can be distinguished from non-specific when the dissociation constant (K_{D}) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding. “Affinity” is defined as the strength of the binding interaction of two molecules, such as an antigen and its antibody, which is defined for antibodies and other molecules with more than one binding site as the strength of binding of the ligand at one specified binding site. Although the noncovalent attachment of a ligand to antibody is typically not as strong as a covalent attachment, “high affinity” is for a ligand that binds to an antibody or other molecule having an affinity constant (Kₐ) of greater than 10⁴ M⁻¹, typically 10⁵-10¹¹ M⁻¹; as determined by inhibition ELISA or an equivalent affinity determined by comparable techniques, such as Scatchard plots or using K_{d}/dissociation constant, which is the reciprocal of the Kₐ, etc.

In one embodiment, the antibody, antigen-binding fragment, or affinity tag binds its target with a K_{D} of 0.1 nM-10 mM. In one embodiment, the antibody, antigen-binding fragment, or affinity tag binds its target with a K_{D} of 0.1 nM-1 mM. In one embodiment, the antibody, antigen-binding fragment, or affinity tag binds its target with a K_{D} within the 0.1 nM range. In one embodiment, the antibody, antigen-binding fragment, or affinity tag binds its target with a K_{D} of 0.1-2 nM. In another embodiment, the antibody, antigen-binding fragment, or affinity tag binds its target with a K_{D} of 0.1-1 nM. In another embodiment, the antibody, antigen-binding fragment, or affinity tag binds its target with a K_{D} of 0.05-1 nM. In another embodiment, the antibody, antigen-binding fragment, or affinity tag binds its target with a K_{D} of 0.1-0.5 nM. In another embodiment, the antibody, antigen-binding fragment, or affinity tag its target with a K_{D} of 0.1-0.2 nM. In some embodiments, the antibody, antigen-binding fragment, or affinity tag bind its target directly. In some embodiments, the antibody, antigen-binding fragment, or affinity tag bind its target indirectly, for example, the antibody, antigen-binding fragment, or affinity tag is a secondary antibody that binds to an antibody bound to the target.

The word “label” as used herein refers to a compound or composition which is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition, which is detectable.

As used herein, the term “probe” refers to synthetic or biologically produced nucleic acids that are engineered to contain specific nucleotide sequences which hybridize under stringent conditions to target nucleic acid sequences.

As used herein, a “labeled probe,” “nucleic acid probe operably linked to a detectable label,” or “nucleic acid strand operably linked to a detectable label” refers to a probe which is prepared with a marker moiety or “detectable label” for detection. The marker moiety is attached at either the 5′ end, the 3′ end, internally, or in any possible combination thereof. That is, one probe may be attached to multiple marker moieties. The preferred moiety is an identifying label such as a fluorophore. The labeled probe may also be comprised of a plurality of different nucleic acid sequences each labeled with one or more marker moieties. Each of the marker moieties may be the same or different. It may be beneficial to label the different probes (e.g., nucleic acid sequences) each with a different marker moiety. This can be accomplished by having a single distinguishable moiety on each probe. For example, probe A may be attached to moiety X and probe B may be attached to moiety Y. Alternatively, probe A may be attached to moieties X and Y while probe B may be attached to moiety Z and W. As another alternative, probe A may be attached to moieties X and Y while probe B may be attached to moieties Y and Z. All the probes “A” and “B” described above would be distinguishable and uniquely labeled.

By “tissue sample” is meant a collection of similar cells obtained from a tissue of a subject or patient, preferably containing nucleated cells with chromosomal material. The four main human tissues are (1) epithelium; (2) the connective tissues, including blood vessels, bone and cartilage; (3) muscle tissue; and (4) nerve tissue. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. The tissue sample may contain compounds which are not naturally intermixed with the tissue in nature such as preservatives, anticoagulants, buffers, fixatives, nutrients, antibiotics, or the like.

For the purposes herein a “section” of a tissue sample is meant a single part or piece of a tissue sample, e.g., a thin slice of tissue or cells cut from a tissue sample. It is understood that multiple sections of tissue samples may be taken and subjected to analysis.

As used herein, “cell line” refers to a permanently established cell culture that will proliferate given appropriate fresh medium and space.

### Detection Methods

In various aspects, provided herein are methods of detecting or locating a target in a biological sample. Targets are detected by contacting a biological sample with a target detection reagent, e.g., an antibody or fragment thereof, and a labeling reagent. The presence or absence of targets are detected by the presence or absence of the labeling reagent, and the location of the labeling reagent indicates where the target biomolecules were located. In some instances, the biological sample is contacted with the target detection reagent and the labeling reagent concurrently e.g., the detection reagent is a primary antibody and the labeling reagent is a fluorescent dye both of which are conjugated to a single nucleic acid strand. Alternatively, the biological sample is contacted with the target detection reagent and the labeling reagent sequentially, e.g., the detection reagent is a primary antibody and the labeling reagent includes a secondary antibody. For example, the biological sample is incubated with the detection reagent, in some cases together with the labeling reagent, under conditions that allow a complex between the detection reagent (and labeling reagent) and target to form. After complex formation the biological sample is optionally washed one or more times to remove unbound detection reagent (and labeling reagent). When the biological sample is further contacted with a labeling reagent that specifically binds the detection reagent that is bound to the target, the biological sample can optionally be washed one or more times to remove unbound labeling reagent. The presence or absence of the target, and if present its location, in the biological sample is then determined by detecting the labeling reagent.

The methods described herein provide for the detection of multiple targets in a sample.

Multiple targets are identified by contacting the biological sample with additional detection reagents followed by additional labeling reagent specific for the additional detection reagents using the methods described above. For example, each target is associated with an affinity tag operably linked to a nucleic acid with a sequence specific or barcode for that target. In some cases, sets or subsets of labeled probes are prepared with distinct labels, e.g., fluorophores that are distinguished by their emission spectra, e.g., one that emits in the green spectra and one that emits in the red spectra. The labeled probes can then be added simultaneously to the biological sample to detect multiple targets at once. Alternatively, sets or subsets of labeled probes are prepared with the same label. Each of the labeled probes can then be added sequentially to detect a specific target, with each labeled probe removed from the biological sample prior to the addition of the next labeled probe to detect multiple targets sequentially.

The detection moiety, i.e., detectable label, is a substance used to facilitate identification and/or quantitation of a target. Detection moieties are directly observed or measured or indirectly observed or measured. Detection moieties include, but are not limited to, radiolabels that can be measured with radiation-counting devices; pigments, dyes or other chromogens that can be visually observed or measured with a spectrophotometer; spin labels that can be measured with a spin label analyzer; and fluorescent moieties, where the output signal is generated by the excitation of a suitable molecular adduct and that can be visualized by excitation with light that is absorbed by the dye or can be measured with standard fluorometers or imaging systems, for example. The detection moiety can be a luminescent substance such as a phosphor or fluorogen; a bioluminescent substance; a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The detection moiety may also take the form of a chemical or biochemical, or an inert particle, including but not limited to colloidal gold, microspheres, quantum dots, or inorganic crystals such as nanocrystals or phosphors. The term detection moiety or detectable label can also refer to a “tag” or hapten that can bind selectively to a labeled molecule such that the labeled molecule, when added subsequently, is used to generate a detectable signal. For instance, one can use biotin, iminobiotin or desthiobiotin as a tag and then use an avidin or streptavidin conjugate of horseradish peroxidase (HRP) to bind to the tag, and then use a chromogenic substrate (e.g., tetramethylbenzidine) or a fluorogenic substrate such as Amplex Red or Amplex Gold (Molecular Probes, Inc.) to detect the presence of HRP. Similarly, the tag can be a hapten or antigen (e.g., digoxigenin), and an enzymatically, fluorescently, or radioactively labeled antibody can be used to bind to the tag. Numerous labels are known by those of skill in the art and include, but are not limited to, particles, fluorescent dyes, haptens, enzymes and their chromogenic, fluorogenic, and chemiluminescent substrates, and other.

A fluorophore is a chemical moiety that exhibits an absorption maximum beyond 280 nm, and when covalently attached in a labeling reagent retains its spectral properties. Fluorophores include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine, a carbocyanine, a carbostyryl, a porphyrin, a salicylate, an anthranilate, an azulene, a perylene, a pyridine, a quinoline, a borapolyazaindacene, a xanthene, an oxazine or a benzoxazine, a carbazine, a phenalenone, a coumarin, a benzofuran and benzphenalenone and derivatives thereof. As used herein, oxazines include resorufins, aminooxazinones, diaminooxazines, and their benzo-substituted analogs.

When the fluorophore is a xanthene, the fluorophore may be a fluorescein, a rhodol, or a rhodamine. As used herein, fluorescein includes benzo- or dibenzofluoresceins, seminaphthofluoresceins, or naphthofluoresceins. Similarly, as used herein rhodol includes seminaphthorhodafluors. Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position. Fluorophores include xanthene (rhodol, rhodamine, fluorescein and derivatives thereof) coumarin, cyanine, pyrene, oxazine and borapolyazaindacene. In addition, the fluorophore can be sulfonated xanthenes, fluorinated xanthenes, sulfonated coumarins, fluorinated coumarins and sulfonated cyanines. The choice of the fluorophore in the labeling reagent will determine the absorption and fluorescence emission properties of the labeling reagent. Physical properties of a fluorophore label include spectral characteristics (absorption, emission and stokes shift), fluorescence intensity, lifetime, polarization and photo-bleaching rate all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

Preferably the detection moiety is a fluorescent dye. Fluorescent dyes include, for example, Fluorescein, Rhodamine, Texas Red, Cy2, Cy3, Cy5, Cy0, Cy0.5, Cy1, Cy1.5, Cy3.5, Cy7, VECTOR Red, ELF™ (Enzyme-Labeled Fluorescence), FluorX, Calcein, Calcein-AM, CRYPTOFLUOR™'S, Orange (42 kDa), Tangerine (35 kDa), Gold (31 kDa), Red (42 kDa), Crimson (40 kDa), BHMP, BHDMAP, Br-Oregon, Lucifer Yellow, Alexa dye family, N-(6-(7-nitrobenz-2-oxa-1,3-diazol-4-yl)amino)caproyl) (NBD), BODIPY™, boron dipyrromethene difluoride, Oregon Green, MITOTRACKER™ Red, DiOC7 (3), DiIC18, Phycoerythrin, Phycobiliproteins BPE (240 kDa) RPE (240 kDa) CPC (264 kDa) APC (104 kDa), Spectrum Blue, Spectrum Aqua, Spectrum Green, Spectrum Gold, Spectrum Orange, Spectrum Red, NADH, NADPH, FAD, Infra-Red (IR) Dyes, Cyclic GDP-Ribose (cGDPR), Calcofluor White, Tyrosine and Tryptophan.

Many of fluorophores can also function as chromophores and thus the described fluorophores are also preferred chromophores.

In addition to fluorophores, enzymes also find use as detectable moieties. Enzymes are desirable detectable moieties because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. This is advantageous where there is a low quantity of target present in the sample or a fluorophore does not exist that will give comparable or stronger signal than the enzyme. However, fluorophores are most preferred because they do not require additional assay steps and thus reduce the overall time required to complete an assay. The enzyme substrate is selected to yield the preferred measurable product, e.g. colorimetric, fluorescent or chemiluminescence. Such substrates are extensively used in the art.

A preferred colorimetric or fluorogenic substrate and enzyme combination uses oxidoreductases such as horseradish peroxidase and a substrate such as 3,3′-diaminobenzidine (DAB) and 3-amino-9-ethylcarbazol-e (AEC), which yield a distinguishing color (brown and red, respectively). Other colorimetric oxidoreductase substrates that yield detectable products include, but are not limited to: 2,2-azino-bis(3-ethylbenzothiaz-oline-6-sulfonic acid) (ABTS), o-phenylenediamine (OPD), 3,3′,5,5′-tetramethylbenzidine (TMB), o-dianisidine, 5-amino salicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplexe Red reagent and its variants and reduced dihydroxanthenes, including dihydrofluoresceins and dihydrorhodamines including dihydrorhodamine 123. Peroxidase substrates that are tyramides represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Additional colorimetric (and in some cases fluorogenic) substrate and enzyme combination use a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy4-methylcoumarinyl phosphate (DiFMUP) fluorescein diphosphate, 3-0-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates.

Glycosidases, in particular β-galactosidase, β-glucuronidase and β-glucosidase, are additional suitable enzymes. Appropriate colorimetric substrates include, but are not limited to, 5-bromo4-chloro-3-indolyl β-D-galactopyranoside (X-gal) and similar indolyl galactosides, glucosides, and glucuronides, o-nitrophenyl β-D-galactopyranoside (ONPG) and p-nitrophenyl β-D-galactopyranosid-e. Preferred fluorogenic substrates include resorufin β-D-galactopyranoside, fluorescein digalactoside (FDG), fluorescein diglucuronide and their structural variants, 4-methylumbelliferyl β-D-galactopyranoside, carboxyumbelliferyl β-D-galactopyranoside and fluorinated coumarin β-D-galactopyranosides.

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful. For example, the enzyme is luciferase or aequorin. The substrates are luciferine, ATP, Ca⁺⁺ and coelenterazine.

In addition to enzymes, haptens such as biotin are useful detectable moieties. Biotin is useful because it can function in an enzyme system to further amplify a detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, or nucleotides.

In some cases, a detectable moiety is a fluorescent protein. Exemplary fluorescent proteins include green fluorescent protein (GFP), the phycobiliproteins and the derivatives thereof, luciferase or aequorin. The fluorescent proteins, especially phycobiliprotein, are particularly useful for creating tandem dye labeled labeling reagents. These tandem dyes comprise a fluorescent protein and a fluorophore for the purposes of obtaining a larger stokes shift where the emission spectra is farther shifted from the wavelength of the fluorescent protein's absorption spectra. This is particularly advantageous for detecting a low quantity of a target in a sample where the emitted fluorescent light is maximally optimized, in other words little to none of the emitted light is reabsorbed by the fluorescent protein. For this to work, the fluorescent protein and fluorophore function as an energy transfer pair where the fluorescent protein emits at the wavelength that the fluorophore absorbs at and the fluorphore then emits at a wavelength farther from the fluorescent proteins than could have been obtained with only the fluorescent protein. A particularly useful combination is phycobiliproteins and sulforhodamine fluorophores, or the sulfonated cyanine fluorophores; or the sulfonated xanthene derivatives. Alternatively, the fluorophore functions as the energy donor and the fluorescent protein is the energy acceptor.

### Methods of Visualizing the Detection Moiety Depend on the Label.

In some cases, the sample is illuminated with a wavelength of light selected to give a detectable optical response, and observed with a means for detecting the optical response. Equipment that is useful for illuminating fluorescent compounds of the present invention includes, but is not limited to, hand-held ultraviolet lamps, mercury arc lamps, xenon lamps, lasers and laser diodes. These illumination sources are optically integrated into laser scanners, fluorescent microplate readers or standard or microfluorometers. The degree and/or location of signal, compared with a standard or expected response, indicates whether and to what degree the sample possesses a given characteristic or desired target.

The optical response is optionally detected by visual inspection, or by use of any of the following devices: CCD camera, video camera, photographic film, laser-scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, examination of the sample optionally includes sorting portions of the sample according to their fluorescence response.

When an indirectly detectable label is used then the step of illuminating typically includes the addition of a reagent that facilitates a detectable signal such as colorimetric enzyme substrate. Radioisotopes are also considered indirectly detectable wherein an additional reagent is not required but instead the radioisotope must be exposed to X-ray film or some other mechanism for recording and measuring the radioisotope signal. This can also be true for some chemiluminescent signals that are best observed after expose to film.

The term “subject” refers to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, a concentration, and the like, may encompass variations of ±20% or ±10%, more preferably ±5%, even more preferably ±1%, and still more preferably ±0.1% from the specified value, as such variations are appropriate to perform the disclosed methods.

### Examples

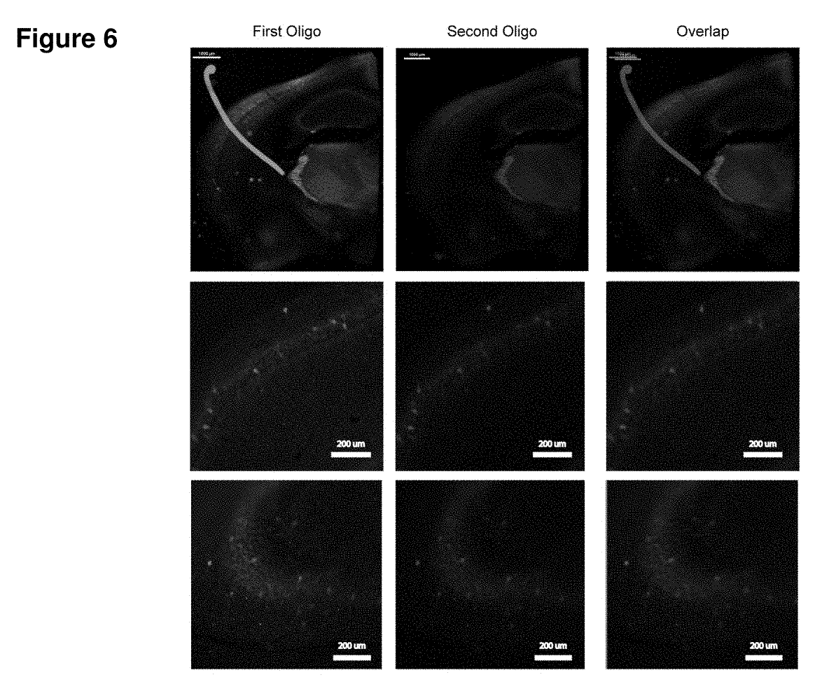

The parvalbumin protein in mouse brain slices was stained with antibodies containing an oligonucleotide barcode as well as a polymerizable handle. A green dye containing oligonucleotide which was complementary to the barcode was added and the tissue was polymerized and digested (green images, left column of FIG. 6). The gel was washed with formamide to remove the green complement and subsequently reacted with a red dye complementary to the barcode (red images, middle column of FIG. 6). The overlay of the images (right column of FIG. 6) demonstrates the close match in location of the dyes in the two rounds of staining confirming the spatial retention of the barcode as well as its ability to reversibly stain for it. The bright line in the green image is due to a crack in the gel (and therefore a light artifact) rather than the location of the dye.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of labeling a biological sample, the method comprising:
contacting the sample with at least one binding composition under conditions where it selectively recognizes a target biomolecule, wherein the binding composition comprises a double-stranded nucleic acid having a first strand comprising a first sequence and a second strand comprising a second sequence that is complementary to the first sequence, wherein the first strand is operably linked to an affinity tag for the target biomolecule and is operably linked to a first detectable label, and wherein the second strand is operably linked to a polyelectrolyte gel binding moiety;
contacting the sample with a solution comprising monomers of a polyelectrolyte gel;
by free radical polymerization, polymerizing the monomers to form the polyelectrolyte gel and covalently conjugate the polyelectrolyte gel binding moiety to the polyelectrolyte gel;
proteolytically digesting the sample; and
dialyzing the sample in water to expand the polyelectrolyte gel.

2. A method of labeling a biological sample, the method comprising:
contacting the sample with at least one binding composition under conditions where it selectively recognizes a target biomolecule, wherein the binding composition comprises a double-stranded nucleic acid having a first strand comprising a first sequence and a second strand comprising a second sequence that is complementary to the first sequence, wherein the first strand is operably linked to an affinity tag for the target biomolecule and is operably linked to a polyelectrolyte gel binding moiety, and wherein the second stand is operably linked to a first detectable label;
contacting the sample with a solution comprising monomers of a polyelectrolyte gel;
by free radical polymerization, polymerizing the monomers to form the polyelectrolyte gel and covalently conjugate the polyelectrolyte gel binding moiety to the polyelectrolyte gel;
proteolytically digesting the sample; and
dialyzing the sample in water to expand the polyelectrolyte gel.

3. A method of labeling a biological sample, the method comprising:
contacting the sample with at least one binding composition under conditions where it selectively recognizes a target biomolecule, wherein the binding composition comprises a single-stranded nucleic acid comprising a first sequence, wherein the single-stranded nucleic acid is operably linked to (i) an affinity tag for the target biomolecule, and (ii) a polyelectrolyte gel binding moiety, and wherein the affinity tag is operably linked to a first detectable label;
contacting the sample with a solution comprising monomers for the polyelectrolyte gel;
by free radical polymerization, polymerizing the monomers to form the polyelectrolyte gel and covalently conjugate the polyelectrolyte gel binding moiety to the polyelectrolyte gel;
proteolytically digesting the sample; and
dialyzing the sample in water to expand the polyelectrolyte gel.

4. The method of labeling a biological sample according to either claim 1 or claim 2, further comprising the step of:
removing the nucleic acid strand unconjugated to the polyelectrolyte gel after covalently conjugating the polyelectrolyte gel binding moiety to it.

5. The method of labeling a biological sample according to either claim 3 or claim 4, further comprising the step of:
hybridizing a nucleic acid probe operably linked to a detectable label and comprising a probe sequence that is complementary to the sequence of the nucleic acid strand conjugated to the polyelectrolyte gel.

6. A method of labeling a biological sample, the method comprising:
contacting the sample with at least one binding composition under conditions where it selectively recognizes a target biomolecule, wherein the binding composition comprises a double-stranded nucleic acid having a first strand comprising a first sequence and a second strand comprising a second sequence that is complementary to the first sequence, wherein the first strand is operably linked to an affinity tag for the target biomolecule, and wherein the second strand is operably linked to a polyelectrolyte gel binding moiety;
contacting the sample with a solution comprising monomers of a polyelectrolyte gel;
by free radical polymerization, polymerizing the monomers to form the polyelectrolyte gel and covalently conjugate the polyelectrolyte gel binding moiety to the polyelectrolyte gel;
proteolytically digesting the sample;
dialyzing the sample in water to expand the polyelectrolyte gel;
removing the second strand; and
hybridizing a nucleic acid probe operably linked to a detectable label and comprising a probe sequence that is complementary to the first sequence.

7. A method of labeling a biological sample, the method comprising:
contacting the sample with at least one binding composition under conditions where it selectively recognizes a target biomolecule, wherein the binding composition comprises a single-stranded nucleic acid comprising a first sequence, wherein the single-stranded nucleic acid is operably linked to (i) an affinity tag for the target biomolecule, and (ii) a polyelectrolyte gel binding moiety;
contacting the sample with a solution comprising monomers of a polyelectrolyte gel;
by free radical polymerization, polymerizing the monomers to form the polyelectrolyte gel and covalently conjugate the polyelectrolyte gel binding moiety to the polyelectrolyte gel;
proteolytically digesting the sample;
dialyzing the sample in water to expand the polyelectrolyte gel; and
hybridizing a nucleic acid probe operably linked to a detectable label and comprising a probe sequence that is complementary to the first sequence.

8. The method according to any one of claims 1-7, wherein each nucleic acid strand independently comprises DNA or RNA.

9. The method according to any one of claims 1-8, wherein the affinity tag comprises an antibody or an antigen-binding fragment.

10. The method of claim 9, wherein the antibody is a secondary antibody.

11. The method according to any one of claims 1-10, wherein the polyelectrolyte gel binding moiety is a methacryloyl group.

12. The method according to any one of claims 1-11, wherein the monomer solution comprises sodium acrylate, acrylamide, and N—N′-methylenebisacrylamide.

13. The method according to any one of claims 1-12, wherein free radical polymerization is induced with ammonium persulfate (APS) initiator and tetramethylethylenediamine (TEMED).

14. The method according to any one of claims 1-13, wherein the biological sample is chemically fixed and permeabilized prior to contact with the binding composition.

15. The method according to any one of claims 1-14, wherein the first detectable label is a fluorescent label.

16. The method according to any one of claims 5-15, wherein the detectable label operably linked to the nucleic acid probe is a fluorescent label.

17. A method of imaging a biological sample, the method comprising:
labeling the sample according to the method of any one of claims 1-16; and
obtaining an image of the sample after expanding the polyelectrolyte gel.

18. The method of claim 17, wherein obtaining the image of the sample comprises detecting the first detectable label.

19. The method of claim 17, wherein obtaining the image of the sample comprises detecting the detectable label operably linked to the nucleic acid probe.

20. The method according to any one of claims 17-19, further comprising the step of:
obtaining an image of the sample before expanding the polyelectrolyte gel.

21. The method of claim 20, wherein obtaining the image of the sample before expansion comprises detecting the first detectable label.

22. The method according to any one of claims 17-21, wherein the image(s) is/are obtained by confocal microscopy.

23. A method of analyzing a biological sample, the method comprising:
(a) contacting the sample with a set of adapters that selectively recognize a set of target biomolecules under conditions where the adapters selectively recognize the target biomolecules, wherein each adapter comprises a double stranded nucleic acid comprising a first strand operably linked to an affinity tag specific for one of the target biomolecules and having a sequence specific for that target biomolecule, and a second strand comprising a sequence specific for that target biomolecule and operably linked to a polyelectrolyte gel binding moiety;
(b) contacting the sample with a solution comprising monomers of a polyelectrolyte gel;
(c) by free radical polymerization, polymerizing the monomers to form the polyelectrolyte gel and covalently conjugate the polyelectrolyte gel binding moiety to the polyelectrolyte gel;
(d) proteolytically digesting the sample;
(e) dialyzing the sample in water to expand the polyelectrolyte gel; and
(f) for each target biomolecule, hybridizing a nucleic acid probe operably linked to a fluorescent label and comprising a probe sequence complementary to the sequence specific for that target biomolecule, and detecting the fluorescent label.

24. A method of analyzing a biological sample, the method comprising:
(a) contacting the sample with a set of adapters that selectively recognize a set of target biomolecules under conditions where the adapters selectively recognize the target biomolecules, wherein each adapter comprises a single-stranded nucleic acid operably linked to a polyelectrolyte gel binding moiety and to an affinity tag for one of the target biomolecules and having a sequence specific for that target biomolecule;
(b) contacting the sample with a solution comprising monomers of a polyelectrolyte gel;
(c) by free radical polymerization, polymerizing the monomers to form the polyelectrolyte gel and covalently conjugate the polyelectrolyte gel binding moiety to the polyelectrolyte gel;
(d) proteolytically digesting the sample;
(e) dialyzing the sample in water to expand the polyelectrolyte gel; and
(f) for each target biomolecule, hybridizing a nucleic acid probe operably linked to a fluorescent label and comprising a probe sequence complementary to the sequence specific for that target biomolecule, and detecting the fluorescent label.

25. The method of analyzing a biological sample according to either claim 23 or claim 24, wherein the fluorescent label operably linked to the nucleic acid probe for a plurality of the target biomolecules includes a fluorophore that is common for that plurality of targets, and each nucleic acid probe of that plurality is hybridized separately from the other probes of that plurality and is removed following detection of that label.

26. The method of analyzing a biological sample according to claim 25, wherein the plurality of target biomolecules is the set of target biomolecules.

27. The method according to any one of claims 23-26, wherein the nucleic acid probe comprises DNA.

28. The method according to any one of claims 23-27, wherein the affinity tag comprises an antibody or an antigen-binding fragment.

29. The method of claim 28, wherein the antibody is a secondary antibody.

30. The method according to any one of claims 23-29, wherein the fluorescent label is detected by confocal microscopy.
